(19) United States
(12) Patent Application Publication
(10) Pub. No.: US 2024/0286082 A1
(43) Pub. Date: Aug. 29, 2024

(54) HOLLOW-FIBRE MEMBRANE FILTER HAVING IMPROVED SEPARATION PROPERTIES

(71) Applicant: FRESENIUS MEDICAL CARE DEUTSCHLAND GMBH, Bad Homburg (DE)

(72) Inventors: Paul GASTAUER, Saarbruecken (DE); Franz KUGELMANN, St. Wendel (DE); Michael PAUL, Lebach (DE); Andreas RUFFING, Bexbach (DE); Tobias VEIT, Oberthal (DE)

(73) Assignee: FRESENIUS MEDICAL CARE DEUTSCHLAND GMBH, Bad Homburg (DE)

(21) Appl. No.: 18/289,373

(22) PCT Filed: May 10, 2022
(86) PCT No.: PCT/EP2022/062581

§ 371 Date: Nov. 3, 2023

(30) Foreign Application Priority Data
May 11, 2021 (DE) 10 2021 112 315.1

(51) Int. Cl. B01D 63/02; A61M 1/16; B01D 69/08

(52) U.S. Cl. CPC B01D 63/024; B01D 69/08; A61M 1/1621; B01D 2313/10; B01D 2313/12; B01D 2313/205

(57) ABSTRACT

The invention relates to a hollow fiber membrane filter for purifying liquids with improved separation properties, comprising a cylindrical housing, first inflow or outflow spaces and second inflow or outflow spaces, each of which surrounds a first and a second end region of the cylindrical housing, the cylindrical housing being embodied in at least one end region such that improved flow of a liquid against the hollow fiber membranes in the interior of the cylindrical housing can take place.

The present invention relates to a hollow fiber membrane filter for purifying liquids, particularly for purifying blood.

### BACKGROUND

Hollow fiber membrane filters are used in the purification of liquids. In particular, hollow fiber membrane filters are used in medical technology for the treatment and decontamination of water and in the treatment of patients with kidney damage through extracorporeal blood therapy in the form of dialyzers or hemofilters. The hollow fiber membrane filters generally consist of a cylindrical housing and a plurality of hollow fiber membranes arranged therein, which are potted at the end of the housing with a potting compound in a potting zone and are connected to the housing in a sealing manner. It is known that such hollow fiber membrane filters are designed for use in a so-called dead-end process or in a cross-flow process with two liquids, so that a mass transfer can take place via the membrane wall of the hollow fiber membranes and a desired purification of the liquid or of one of the liquids takes place. For this purpose, the hollow fiber membrane filters are designed in such a way that the lumina of the hollow fiber membranes form a first flow space through which a first liquid flows, and the spaces between the hollow fiber membranes in the housing of the hollow fiber membrane filter form a second flow space through which a second liquid can flow. Inflow or outflow spaces having liquid access points for conducting the first and second liquid into and out of the respective flow spaces of the hollow fiber membrane filter are disposed in one or both end regions of the hollow fiber membrane filters.

A multitude of hollow fiber membrane filters exist on the market which have different designs particularly in terms of the construction of the end regions and their inflow or outflow spaces connecting to the end. With regard to the development of hollow fiber membrane filters for extracorporeal blood treatment (dialyzers and hemofilters), ongoing attempts are being made to change and improve the design of the hollow fiber membrane filters. Among other things, the focus is on ensuring that the geometry of the inflow or outflow spaces of a hollow fiber membrane filter through which blood flows enable it to flow through the spaces as gently as possible, so that turbulent flows or stagnant flows that can damage the blood cells are avoided. As is generally customary in extracorporeal blood purification, the hollow fiber membrane filters are designed in such a way that the patient's blood is conducted through the first flow space—i.e., through the lumina of the hollow fiber membranes.

What is more, a multitude of design proposals exist among commercially available hollow fiber membrane filters for extracorporeal blood treatment that are intended to improve the flow against the hollow fiber membranes in the second flow space. During the therapeutic use of hollow fiber membrane filters for extracorporeal blood treatment, an aqueous, physiologically compatible liquid (dialysis liquid) usually flows through the second flow space. The removal of harmful metabolites from the patient's blood then takes place by means of transmembrane mass transfer. The flow against the hollow fiber membranes in the second flow space, among other things, is crucial for improved separation of the metabolites.

Kunikata et al. (Kunikata; ASAIO Journal, 55 (3), pp. 231-235 (2009), assess the performance data of various commercially available dialyzers with regard to their different designs in the inflow region for the dialysis fluid. In this publication, various design models are shown which are intended to bring about a favorable flow behavior of the dialysis fluid entering the dialyzer. In particular, such solutions are shown according to which the dialysis fluid flowing in via the dialysate connection is intended to flow evenly around the hollow fiber membranes that are arranged in the cylindrical housing in the end region of a dialyzer, thus enabling a uniform flow against the hollow fiber membranes to occur. The Asahi Kasei Kuraray APS-15S and Nipro PES-150S dialyzers shown in Kunikata are equipped with a partially circumferential baffle plate opposite the dialysate connection. The Asahi Kasei Kuraray APS-15SA dialyzer has a circumferential baffle plate over which the incoming dialysis fluid flows. The Tory CS-16U dialyzer has a circumferential baffle plate with slots through which the incoming dialysis fluid flows. The FPX140 dialyzer from Fresenius exhibits a design in which the hollow fiber membranes in the end region of the dialyzer are framed by a crenelated structure. Based on the investigations, Kunikata et al. come to the conclusion that the design of the dialyzers shown in the end region of the dialyzers can improve the flow of dialysis fluid against the hollow fiber membranes, so that the performance data of the hollow fiber membrane filters shown can be improved.

The embodiments shown in Kunikata have an elaborate housing design, so that these designs must be regarded as disadvantageous with regard to the high level of productivity that is aspired to on a large scale. In addition, there is a continuous search for ways to simplify and accelerate the production of hollow fiber membrane filters. Means are therefore being sought which, in particular, make it possible to manufacture the hollow fiber membrane filters through rational manufacturing steps.

### OBJECT OF THE INVENTION

It was therefore the object of the invention to provide a hollow fiber membrane filter having improved flow against the hollow fiber membranes and, as a consequence, improved performance data.

### SUMMARY OF THE INVENTION

This object is achieved by a hollow fiber membrane filter with the features of claim 1. Claims 2 to 12 relate to preferred embodiments.

### DETAILED DESCRIPTION OF THE INVENTION

The invention relates to the invention a hollow fiber membrane filter having a cylindrical housing that extends along a central axis in the longitudinal direction, with a housing interior space, a first end region with a first end, and a second end region with a second end, a plurality of hollow fiber membranes that are arranged in the cylindrical housing and embedded in a sealing manner in the first end region and in the second end region of the cylindrical housing in a respective potting compound in a potting zone, the ends of the hollow fiber membranes being open so that the lumina the hollow fiber membranes form a first flow space and the housing interior space surrounding the hollow fiber membranes forms a second flow space,
first inflow or outflow spaces, each adjoining with their front end the first and second end of the cylindrical housing and the potting zone, which are in fluid communication with the first flow space of the hollow fiber membrane filter and each of which has first liquid access points for conducting liquid into/out of the first inflow or outflow space,
second inflow or outflow spaces surrounding the first and the second end region of the cylindrical housing which are in fluid communication with the second flow region and each of which has second liquid access points for conducting liquid into/out of the second inflow or outflow space,
seals that separate the first inflow or outflow space from the second inflow or outflow space, passage openings in the end regions of the housing that form a fluid connection between the second inflow or outflow space and the second flow space, characterized in that
in at least one end region of the cylindrical housing, the ratio of the sum of the flow cross sections of all passage openings to the flow cross section of the at least one second inflow or outflow space is between 0.5:1 to 7:1, or 0.75:1 to 5:1, or 1:1 to 3:1.

The hollow fiber membrane filter of the aforementioned type has high performance parameters with regard to the purification of liquids. It is assumed that, according to the definition given above, an improved flow of a liquid against the hollow fiber membrane is able to occur in at least one end region of the hollow fiber membrane filter as a result of a liquid flowing through a second connection into the second inflow or outflow space and through the passage openings in the end region of the cylindrical housing into the second flow space. In particular, improved separation performance of the test solutes urea and vitamin B12 has been measured for the hollow fiber membrane filters according to the invention. The clearance is determined in accordance with the DIN/EN/ISO 8637:2014 standard.

In one embodiment, the hollow fiber membrane filter can be embodied as a dialyzer. In terms of the present application, the term “dialyzer” is use to represent blood filter devices that are based on the structure of a hollow fiber membrane filter, such as a dialysis filter or a hemofilter. In other applications, the hollow fiber membrane filter according to the invention can also be used as a filter for water treatment. The structure of hollow fiber membranes is inherently known in the prior art.

The term “end region of the cylindrical housing” is to be understood in the context of the present application as a section on the cylindrical housing that extends from the end of the housing to the center of the cylindrical housing. The term “end region” indicates that it is an area on the cylindrical housing that takes up only a small area compared to the longitudinal extension of the cylindrical housing. In particular, one of these end regions takes up less than one fifth, or less than one eighth, or less than one tenth, or less than one fifteenth of the total length of the cylindrical housing.

The potting zone is located in a part of the end region of the cylindrical housing. In the context of the present application, the “potting zone” is the region in which the hollow fiber membranes of the hollow fiber membrane filter are embedded in a potting compound. The hollow fiber membranes are embedded in the potting compound in such a way that they are fixed to the end regions of the cylindrical housing. The potting compound forms a seal with the end region of the cylindrical housing. In particular, a provision is made that the potting zone takes up less than three quarters, or less than two thirds, or less than half the width of the end region. The potting compound is plate-shaped and arranged in the cylindrical housing perpendicular to the central axis of the cylindrical housing. The term “central axis” is to be understood as a longitudinal axis of the cylindrical housing that runs in the center of the cylindrical housing of the hollow fiber membrane filter. In the context of the present application, the term “central axis” is used for the geometric description of the hollow fiber membrane filter.

First inflow or outflow spaces are located on the front side adjacent to the potting zones at the end of the cylindrical housing. In the context of the present application, the term “first inflow or outflow space” is understood to mean a volume area in the hollow fiber membrane filter into which liquid can enter either before it enters the first flow space of the hollow fiber membrane filter or after it has exited the first flow space of the hollow fiber membrane filter. The first inflow and outflow spaces adjoin the potting zone in a sealing manner via a wall of the end caps, and/or they adjoin the end of the end region of the cylindrical housing. In certain embodiments, the first inflow or outflow spaces can be embodied as end caps. The end caps are located at the ends of the cylindrical housing and are connected to the cylindrical housing of the hollow fiber membrane filter in a liquid-tight and positive manner via a wall of the end caps. The first inflow or outflow spaces each have a first liquid access point for conducting liquid into/out of the first inflow or outflow spaces. The first inflow or outflow spaces are therefore in fluid communication with the first flow space of the hollow fiber membrane filter, which is formed by the lumina of the hollow fiber membranes. In the context of the present application, “lumina” or “lumen” is understood to mean the cavity of the hollow fiber membranes.

According to the invention, the hollow fiber membrane filter also has second inflow or outflow spaces that surround the respective end regions of the cylindrical housing. In the context of the present application, the term “second inflow or outflow spaces” is understood to mean a delimited volume area in the hollow fiber membrane filter into which liquid can enter either before it enters the second flow space of the hollow fiber membrane filter or after it has exited the second flow space of the hollow fiber membrane filter. The second inflow or outflow spaces are each formed by casings that enclose the end regions of the cylindrical housing. A wall of the casings sealingly adjoins the potting zone and/or the end of the end region of the cylindrical housing. The casings can be part of the cylindrical housing and attached thereto, in which case the second inflow or outflow spaces are sealingly enclosed by the casing. Alternatively, the casing can also be formed by separate sleeves or as part of end caps that also enclose the first inflow or outflow spaces. The end caps are then designed such that they sit positively on the ends of the cylindrical housing, adjoin the housing in a liquid-tight manner, and, at the same time, also form the casing of the second inflow or outflow spaces. The second inflow or outflow spaces each have a second liquid access point for conducting liquid into/out of the second inflow or outflow spaces. The second inflow or outflow spaces are in fluid communication with the second flow space of the hollow fiber membrane filter, which second flow space is formed by the housing interior space of the hollow fiber membrane filter surrounding the hollow fiber membranes.

As described above, the first and second inflow or outflow spaces sealingly adjoin the potting zone and/or the end of the end region of the cylindrical housing. The first and second inflow or outflow spaces are therefore separated from one another in a liquid-tight manner at this location. Some examples of suitable sealing means include O-rings, welding zones, or bonding zones that are arranged between the ends of the end region of the cylindrical housing or of the potting compounds and the wall of the first and second inflow or outflow spaces.

A fluid connection between the second inflow or outflow spaces and the second flow space is formed via the passage openings in the end region of the cylindrical housing. Liquid can thus enter the second flow space or be discharged from the second flow space. The number of passage openings in an end region of the cylindrical housing can be at least 5, or 10, or 15, or 20, or 30, or 40 or 60. The number of passage openings is at most 350, or 300, or 250, or 200, or 180, or 150. The number of passage openings in an end region of the cylindrical housing is preferably between 10 and 350, or between 10 and 40, or between 15 and 300, or between 20 and 250, or between 30 and 200 or between 40 and 180 or between 60 and 180.

The geometric ratio of the sum of the flow cross sections of all passage openings to the flow cross section of the at least one second inflow or outflow space is between 0.5:1 to 7:1, or 0.75:1 to 5:1, or 1:1 to 3:1. The “sum of the flow cross sections of the passage openings” is understood to mean the sum of the surface areas of all of the individual passage openings in an end region of the cylindrical housing.

In the context of the present application, the “flow cross section of a second inflow or outflow space” is understood to mean the cross-sectional area of the second inflow or outflow space that is created through formation of a cross section through the hollow fiber membrane filter and through the central axis of the cylindrical housing. The cross section is placed in such a way that the second liquid access points at the second inflow and outflow spaces are not touched. If two cross-sectional areas of the second inflow or outflow space are mapped in the aforementioned cross-sectional view, with the second inflow or outflow spaces having a rotationally symmetrical geometry, for example, only one of these cross-sectional areas is used to determine the flow cross section.

Since improved flow to the hollow fiber membranes in the second flow space is considered to be the cause of the above-described effect, it is sufficient if the geometric ratio of the flow cross section of the passage openings to the flow cross section of the at least one second inflow or outflow space is fulfilled in only one end region of the cylindrical housing, where, as a result of the use of the hollow fiber membrane filter, the hollow fiber membranes in the second flow space are flowed against by the incoming liquid from the second inflow or outflow space.

In an advantageous embodiment of the invention, the hollow fiber membrane filter is characterized in that, in the at least one end region of the cylindrical housing in which the defined ratio of the sum of the flow cross sections of all passage openings to the flow cross section of the at least one second inflow or outflow space is present, the at least one second inflow or outflow space, starting from the second liquid access point, forms a circumferential space, particularly an annular gap, that is rotationally symmetrical to the central axis of the cylindrical housing. By virtue of the rotationally symmetrical geometry of the second inflow or outflow spaces, the components for the hollow fiber membrane filter can be manufactured in a process-optimized manner, particularly using injection molding techniques.

In another advantageous embodiment of the invention, the hollow fiber membrane filter is characterized in that the two second inflow or outflow spaces form the rotationally symmetrical circumferential space defined in claim, particularly the annular gap, and that, furthermore, the ratio of the sum of the flow cross sections of all passage openings in both end regions of the cylindrical housing to the flow cross section of the at least one second inflow or outflow space lies in the defined range between 0.5:1 to 7:1, or 0.75:1 to 5:1, or 1:1 to 3:1. According to this condition, the hollow fiber membrane filter has a symmetrical construction in the end regions of the cylindrical housing. In particular, the symmetrical structure simplifies the production of the hollow fiber membrane filter, since the number of different components is smaller and no preferred orientations of the components need to be observed during the manufacturing process. The same also applies to the use of hollow fiber membrane filters in filtration applications. Here as well, it is advantageous if the hollow fiber membrane filter has a symmetrical construction, so that no preferred orientation needs to be adhered to in the application.

In another embodiment, the hollow fiber membrane filter is characterized in that the at least one end region, and optionally the second end region, is divided into a proximal end region, a distal end region, and a transition region disposed between the proximal and distal end regions, wherein one end of the distal end region is the end of the cylindrical housing, and the distal end region has an inner diameter that is at least 2% larger than the inner diameter of the proximal end region. In terms of this embodiment, the proximal end region is proximal to the center of gravity of the cylindrical housing. Accordingly, the distal end region is arranged distal to this center of gravity of the cylindrical housing and is thus located at the ends of the cylindrical housing. Advantageously, the packing density of the hollow fiber membranes arranged in the cylindrical housing of the hollow fiber membrane filter is reduced in the distal end region due to the larger inner diameter of the cylindrical housing in this part of the end region. This offers the advantage that fewer defect points occur when the hollow fiber membrane is cast in the cylindrical housing during the manufacture of the hollow fiber membrane filter. Furthermore, the lower packing density in this distal end region makes the hollow fiber membranes more amenable to flow by dialysis fluid.

In the transition region of the end region, the inner diameter of the cylindrical housing increases by more than 2%. Preferably, the inner diameter of the cylindrical housing increases in the transition region by more than 3%, or more than 4%, or more than 5% and at most by 10%, or at most by 8%, or at most by 7%, or at most by 6%, in particular by 2 to 10%, or 3 to 8%, or 4 to 7%. The transition region occupies at least 1/10, or at least 1/12, or at least 1/14, or at least 1/15, or at least 1/17, or at least 1/18, or at least 1/20 and at most 1/40, or at most 1/35, or at most 1/30 or at most 1/25, in particular 1/10 to 1/40, or 1/12 to 1/35, or 1/14 to 1/30, or 1/15 to 1/25, of the total length of the cylindrical housing in the direction of extension of the center axis of the cylindrical housing.

In a further embodiment of the aforementioned embodiment, the hollow fiber membrane filter is characterized in that the passage openings are arranged at the distal end region. The dialysis fluid entering the second flow chamber can thus be passed directly via the passage openings into that part of the hollow fiber membranes which have a lower packing density. This results in an advantageous circumferentially uniform flow to the hollow fiber membranes in the distal end region, which can also better penetrate the arrangement of hollow fiber membranes due to the lower packing density in this part of the end region before the flow of dialysis fluid enters the part of the hollow fiber membranes with a higher packing density.

In another advantageous embodiment of the invention, the hollow fiber membrane filter is characterized in that the passage openings are circular, oval-, or slot-shaped. Depending on the different inner diameters of the cylindrical housing, which are provided for different applications, the number and shape of the passage openings in the end region of the cylindrical housing can vary. This also depends on the manufacturing possibilities of the cylindrical housing, which is preferably manufactured using injection molding technology. It is therefore advantageous to arrange a multitude of passage openings having a circular, oval-, or slot-shaped shape in the end region of the cylindrical housing.

In another advantageous embodiment of the invention, the hollow fiber membrane filter is characterized in that the passage openings are arranged on isolated and/or opposite sections or evenly around the circumference in the end region of the cylindrical housing.

In another advantageous embodiment of the invention, the hollow fiber membrane filter is characterized in that the sum of the flow cross sections of all passage openings is 10 to 350 mm², or 15 to 200 mm², or 15 to 150 mm², or 20 to 110 mm². The envisaged sum of the flow cross sections of all passage openings is dependent on the inner diameter of the cylindrical housing of the hollow fiber membrane filter and, consequently, on the number of hollow fiber membranes. Hollow fiber membrane filters with a larger membrane surface area and a higher number of hollow fiber membranes require a commensurately high flow volume in the second flow space of the hollow fiber membrane filter in order to achieve sufficient filtration performance. In one example, with an arrangement of approx. 10,000 hollow fiber membranes in the second flow space of the hollow fiber membrane filter, the sum of all flow cross sections of the passage openings is in the range of approx. 90 to 150 mm². The inner diameter of the cylindrical housing can be between 28 and 35 mm. In other embodiments, the inner diameter of the housing can be between 20 and 45 mm, particularly between 28 and 45, more particularly between 30 and 40 mm. The adaptation of the sum of all flow cross sections of the passage openings to the inner diameter of the cylindrical housing is used to regulate a defined inflow of liquid into the second flow space and thus to achieve improved flow against the hollow fiber membranes in the second flow space.

In another advantageous embodiment of the invention, the hollow fiber membrane filter is characterized in that the flow cross section of one of the second or two second inflow or outflow spaces is 20 to 50 mm², 20 to 40 mm², or 25 mm². Here, too, the flow cross section of the inflow or outflow spaces can be adapted to the inner diameter of the cylindrical housing of the hollow fiber membrane filter and thus also take on different values for the number of hollow fiber membrane filters. In one example, with an arrangement of approximately 10,000 hollow fiber membranes in the second flow space of the hollow fiber membrane filter, the flow cross section of the inflow or outflow spaces is 20 to 30 mm². The adaptation of the flow cross section of the inflow or outflow spaces to the inner diameter of the cylindrical housing results in an efficient distribution of the liquid flowing into the second inflow or outflow space, so that when the liquid enters the second flow space, a uniform flow against the hollow fiber membranes can be achieved.

The inner diameter of a hollow fiber membrane filter according to the invention can be 20 to 45 mm. In particular, 5000 to 15000 hollow fiber membranes can be arranged in the cylindrical housing of the hollow fiber membrane filter, so that the hollow fiber membrane filter has a membrane surface area of 0.6 to 2.5 m². The “membrane surface area” of the hollow fiber membrane filter is calculated from the product of the inner surface area of the hollow fiber membrane and the number of hollow fiber membranes that are arranged in the cylindrical housing of the hollow fiber membrane filter. The inner surface area of the hollow fiber membrane is calculated from the product of the inner diameter of a hollow fiber membrane, the circle constant π, and the actual effective length. According to the invention, in one embodiment, the actual effective length of the hollow fiber membrane filter in the cylindrical housing is 200 to 350 mm. In the context of the present application, the actual “effective length” of the hollow fiber membrane filter or of the hollow fiber membranes is understood to be the distance between the potting compounds in which an effective exchange of substances can take place via the hollow fiber membranes. In one embodiment, the packing density of the hollow fiber membranes in the hollow fiber membrane filter is 55 to 65%, particularly between 60 and 65%. In the context of the present application, packing density is understood to mean the portion of the housing interior space of the cylindrical housing that is occupied by the hollow fiber membranes. The packing density is the percentage ratio of the sum of the cross-sectional areas of the hollow fiber membranes to the cross-sectional area of the cylindrical housing of the hollow fiber membrane filter, the cross-sectional area of the cylindrical housing only being understood to be the cross-sectional area specified by the inner diameter.

Hollow fiber membranes made of polysulfone and polyvinylpyrrolidone are preferably used to construct a hollow fiber membrane filter according to the invention. In particular, the hollow fiber membranes can have a wave-like shape. Such wave-shaped hollow fiber membranes are described, for example, in WO 01/60477 A2. The amplitude of the wave shape can be from 0.03 to 0.8 mm. The wavelength of the wave shape can be 3 to 30 mm, particularly 5 mm to 12. The diameter of the hollow fiber membranes can be 205 to 330 μm, particularly 170 to 200 μm, with the diameter of the lumen of the hollow fiber membranes being 165 to 230 μm, particularly 175 to 200 μm.

The potting compounds with which the hollow fiber membranes are embedded and sealed in the respective end regions of the cylindrical housing are preferably made of polyurethane.

The cylindrical housing and end caps are preferably made of a polypropylene material.

In an advantageous embodiment of the invention, the hollow fiber membrane filter is constructed in such a way that it has an aspect ratio of 8 to 12, particularly 9 to 11, more particularly 9 to 10. In the context of the present application, the aspect ratio is understood to be the quotient of the actual effective length and the inner diameter of the cylindrical housing of the hollow fiber membrane filter. A flow against the hollow fiber membranes in the second flow region via the ratio of the sum of the flow cross sections of all of the passage openings to the flow cross section of the at least one second inflow or outflow space is thus further improved through a reduction in the inner diameter of the cylindrical housing with the same packing density and membrane surface area. In order to meet these conditions, the hollow fiber membrane filter is constructed according to the invention in such a way that, given the same membrane surface area and packing density, it has a smaller number of hollow fiber membranes but a greater actual effective length. This is particularly advantageous for hollow fiber membrane filters that have a large membrane surface area.

In another advantageous embodiment of the invention, the hollow fiber membrane filter is characterized in that the first and the second inflow or outflow space in the first end region of the cylindrical housing and the first and the second inflow or outflow space in the second end region of the cylindrical housing are respectively enclosed by a first and a second end cap. The end caps are advantageously integrally formed. The end caps are designed in such a way that one wall of the end cap encloses the first inflow or outflow space and another wall forms a casing that encloses the second inflow or outflow space. The end caps are geometrically shaped in such a way that they sit in a positive manner on the end regions of the cylindrical housing and are rendered liquid-tight by seals. The end caps are advantageously manufactured by injection molding. The production of a hollow fiber membrane filter using the end caps defined here contributes to the process-optimized production of the hollow fiber membrane filter. First and second liquid access points are disposed on the end caps.

In another advantageous embodiment of the invention, the hollow fiber membrane filter is characterized in that the first end cap positively adjoins, particularly in a liquid-tight manner, an annular, outer circumferential projection on the first end region of the cylindrical housing. In particular, the second end cap also positively adjoins an annular, outer circumferential projection on the second end region of the cylindrical housing, particularly in a liquid-tight manner. End caps and cylindrical housing are thus connected in a liquid-tight manner along the outer circumferential projection. A seal can be made by welding or gluing.

In another advantageous embodiment of the invention, the hollow fiber membrane filter is characterized in that the first end cap positively adjoins the first end of the cylindrical housing, particularly in a liquid-tight manner, along an inner circumferential circular line. In particular, the second end cap also positively adjoins the second end of the cylindrical housing, particularly in a liquid-tight manner, along an inner circumferential circular line. The inner circumferential circular line can be embodied, for example, as a circular bead or projection on the inside of the end caps. Alternatively, however, the inside of the wall of the end caps can connect directly to the end of the cylindrical housing. The connection of the circular line of the end caps to the ends of the cylindrical housing creates a liquid seal between the first inflow and outflow space and the second inflow and outflow space by means of welding, gluing, or O-rings.

In another advantageous embodiment of the invention, the hollow fiber membrane filter is characterized in that the capacity of one or both of the second inflow or outflow spaces is between 1.5 and 5 cm³. By means of a delimited volume area of the second inflow and/or outflow spaces, it can be ensured, in particular, that the liquid entering the second inflow or outflow spaces can be uniformly distributed as a function of the inner diameter of the cylindrical housing. This also prevents flows from stagnating in regions of the at least one second inflow or outflow space and flowing inhomogenously against the hollow fiber membranes in the second flow region.

In another advantageous embodiment of the invention, the hollow fiber membrane filter is characterized in that the cylindrical housing and the end caps are made of a thermoplastic material, particularly of polypropylene. The cylindrical housing and the end caps can thus be advantageously produced using a process-optimized injection molding process. Furthermore, the selection of the materials also results in the advantage that the cylindrical housing and the end caps can be connected to one another in a positive and sealing manner through a welding process.

### DESCRIPTION OF THE INVENTION ON THE BASIS OF THE FIGURES

FIG. 1a shows a cross section of a hollow fiber membrane filter according to the invention through the central axis A of the cylindrical housing.

FIG. 1b shows another cross section of a hollow fiber membrane filter according to the invention, which runs through both the central axis A of the cylindrical housing and the central axis B of the second liquid access point.

FIG. 2a shows a side view of a cylindrical housing of a hollow fiber membrane filter according to the invention, the end region of the cylindrical housing being depicted.

FIG. 2b shows a side view of another embodiment of a cylindrical housing of a hollow fiber membrane filter according to the invention, wherein the end region of the cylindrical housing is shown. The illustration according to FIG. 2b is provided with dimensioning. The values of the dimensions refer to the unit millimeter (mm).

FIG. 3 shows a schematic representation of a cross section of a commercially available FX60 hollow fiber membrane filter from Fresenius Medical Care Deutschland GmbH, which runs through both the central axis A of the cylindrical housing and the central axis B of the second liquid access point.

FIG. 4 shows a side view of a cylindrical housing of a commercially available FX60 hollow fiber membrane filter from Fresenius Medical Care.

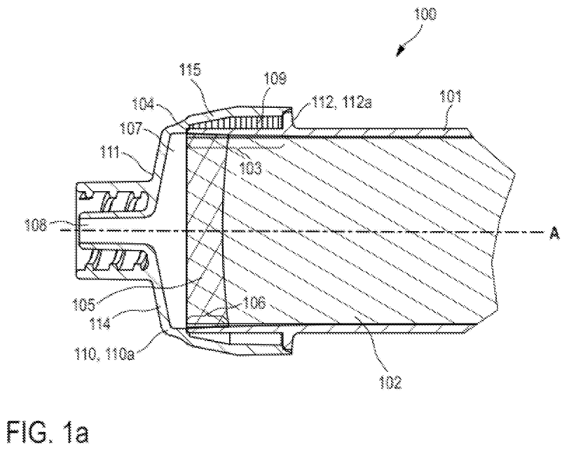

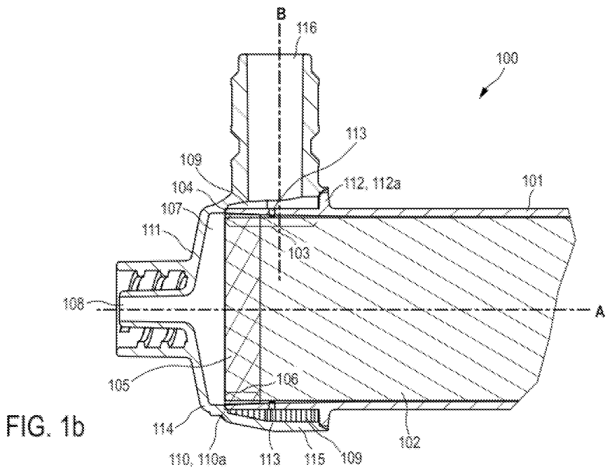

FIG. 1a shows a schematic representation of a cross section of a hollow fiber membrane filter 100 according to the invention along the central axis A of the cylindrical housing 101. Only a portion of the hollow fiber membrane filter is shown in FIG. 1a, which illustrates a first end 104 on the cylindrical housing 101 with a first end region 103. A portion of the end region 103 is occupied by a potting zone 106 in which a potting compound 105 is disposed on the front side relative to the longitudinal orientation, i.e., perpendicular to the central axis A of the cylindrical housing, which potting compound 105 is respectively embedded in hollow fiber membranes (not shown in FIG. 1a) in the housing interior space 102 in the first end region 103 and in the second end region (not shown) of the cylindrical housing 101 so as to form a seal with the housing 101. Also shown is an end cap 111 with a wall 114 that encloses the first inflow or outflow space 107, as well as a casing region 115 that encloses the second inflow or outflow space 109. The surface of the flow cross section of the second inflow or outflow space 109 is indicated by hatching in FIG. 1a. A liquid access point 108 is also shown. In the illustration, the liquid access point 108 exhibits the typical details of a blood connection of a dialyzer. The liquid access point 108 forms a liquid access point to the first inflow or outflow space 107. The end cap 111 shown in FIG. 1 is integrally formed, so that the wall 114 and the casing 115 are part of the end cap. According to the arrangement shown in FIG. 1a, the space of the first and second inflow or outflow spaces (107, 109) is enclosed by the end cap 111, the cylindrical housing 101, and the potting compound 105. The first inflow or outflow space is sealed off at the end 104 of the cylindrical housing 101 by means of a circumferential seal 110. An inner circular circumference 110a of the end cap 111, which is only shown in cross section in FIG. 1, is used for this purpose. In the embodiment shown in FIG. 1, the inner circumference 110a of the end cap 111 sits in a positive manner on the end 104 of the cylindrical housing 101, so that the seal 110 is created between the end 104 of the cylindrical housing and the end cap 111. Liquid that flows into the first inflow or outflow space 107 through the liquid access point 108 flows into the lumina of the hollow fiber membranes and thus into the first flow space exclusively via the open ends of the hollow fiber membranes in the potting compound 105 (not shown in FIG. 1a). Another circumferential liquid seal 112 is created by the annular outer circumferential projection 112a on the cylindrical housing 101 that adjoins the casing 115 of the end cap 111 in a positive and liquid-tight manner.

FIG. 1b shows another cross section of a hollow fiber membrane filter 100 according to the invention, which runs through both the central axis A of the cylindrical housing and the central axis B of the second liquid access point. The central axis B runs centrally in the second liquid access point 116, which adjoins the second inflow or outflow space 109. The designations 100 to 111 and 114 and 115 in FIG. 1b designate the corresponding details from FIG. 1a. The surface of the flow cross section of the second inflow or outflow space 109 is indicated by parallel lines in FIG. 1b. In addition, in this cross-sectional illustration, the passage openings 113 can be seen on opposite sides of the end region 103 of the cylindrical hollow fiber membrane filter. According to FIG. 1b, a fluid connection occurs via the second liquid access point 116 of the second inflow or outflow space 109 and the second flow space in the housing interior space 102 of the hollow fiber membrane filter 100 via the passage openings 113. In an embodiment shown in FIG. 1b, a multitude of passage openings, of which only two are visible in the cross-sectional view of FIG. 1b, are arranged opposite one another on the end region 103 of the cylindrical hollow fiber membrane filter.

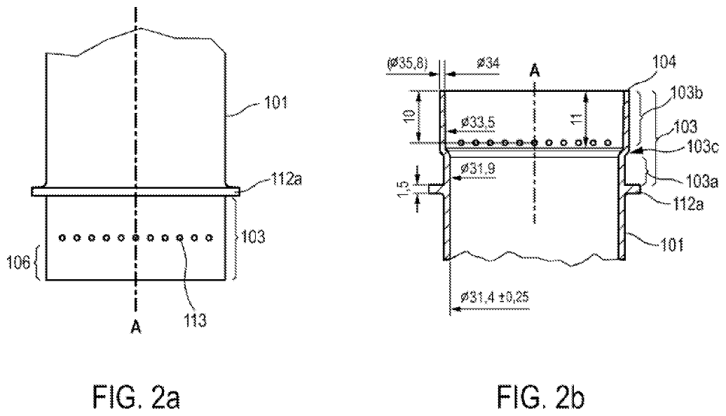

FIG. 2a shows a schematic representation of a portion of a cylindrical housing 101 of a hollow fiber membrane filter according to the invention in a side view. In the illustration of FIG. 2a, the portion with the first end 104 of the cylindrical housing 101 is shown. FIG. 2a also shows the annular, outer circumferential projection 112a on the cylindrical housing 101, which is provided for the purpose of producing a seal 112 on a casing 115 of an end cap 111. Reference 103 denotes the end region of the cylindrical housing 101. Reference 106 denotes the potting zone in the end region, with no potting compound 105 being shown in FIG. 2a. The central axis A indicates the longitudinal orientation of the cylindrical housing. In the side view, a plurality of passage openings 113 is shown, which form in the hollow fiber membrane filter the connection between the second inflow or outflow space 109 and the second flow space (both not shown in FIG. 2a). In the illustration shown, the passage openings are depicted as circular, but they can also have the shape of an oval, slot, or U. The flow cross sections of the passage openings 113 result from the sum of the flow cross sections of all of the individual passage openings 113. The embodiment shown in accordance with FIG. 2a has twenty-two passage openings 113 in the end region 103 of the cylindrical housing 101, of which only half—i.e., 11—are visible in FIG. 2a. An additional eleven passage openings are located on the opposite side of the end region 103 of the cylindrical housing 101.

FIG. 2b shows in schematic view an embodiment of a part of a cylindrical housing 101 of a hollow fiber membrane filter according to the invention in a lateral view. In the illustration of FIG. 2b, the part with the first end 104 of the cylindrical housing 101 is shown. Further shown in FIG. 2b is the annular outer circumferential projection 112a on the cylindrical housing 101, which is provided for making a seal 112 on a casing 115 of an end cap 111 (not shown in FIG. 2b). Further shown in FIG. 2b are 103—the end region of the cylindrical housing 101, the central axis A, 113—circular through openings.

In the embodiment shown, the distance from the center of the passage openings 113 to the end 104 of the cylindrical housing 101 is 10 mm. At the end 104 of the cylindrical housing, the diameter of the opening of the cylindrical housing is 34 mm. In the embodiment shown, the end region 103 of the cylindrical housing is divided into a proximal end region 103a and a distal end region 103b. In the embodiment shown, the proximal end region 103a is disposed adjacent to the annular outer circumferential projection 112a and is thus proximal to a center of gravity of the cylindrical housing in terms of the embodiment shown in FIG. 2b. In the embodiment shown in FIG. 2b, the inner diameter of the distal end region 103b of the cylindrical housing is larger than that of the proximal end region 103a. The proximal end region and the distal end region adjoin each other through a transition region 103c. In the transition region 103c of the end region 103, the inner diameter of the cylindrical housing increases by more than 3%. In particular, according to the embodiment shown in FIG. 2b, the diameter of the distal end region 103b at the end of the cylindrical housing is 34 mm, whereas the inner diameter of the distal end region 103b subsequently at the transition portion 103c is 33.5 mm. The inner diameter of the cylindrical housing 101 at the proximal end region is 31.9 mm in the shown embodiment of FIG. 2b. Accordingly, the increase in inner diameter from the proximal 103a to the distal 103b end region is 1.6 mm in the embodiment shown. The inner diameter of the cylindrical housing 101 is 31.4 mm in a central region. From the dimensions shown in FIG. 2b, it can be seen that the inner diameter in each of the distal 103b end region and the proximal end region 103a is further tapered toward the central portion of the cylindrical housing. The conical shape of the inner diameter of the individual regions of the cylindrical housing 101, illustrated according to FIG. 2b, results from the need to be able to demold the cylindrical housing as an injection molded part from an injection molding machine. Such required geometries of injection molded parts are known in injection molding technology. The change in internal diameter at the transition region 103c must be distinguished from these necessary conically extending changes in internal diameter. The transition region 103c occupies an area of less than 2 mm in the direction of extension of the center axis A in the shown embodiment of FIG. 2b, in which the inner diameter of the proximal end region increases from 31.9 mm to the inner diameter of the distal end region of 33.5 mm. The transition area occupies about only 1/15 of the total length of the cylindrical housing.

In one embodiment of a hollow fiber membrane filter according to the invention, which is worked according to the details shown in FIGS. 1a, 1b and 2, the sum of the flow cross sections of all passage openings can be 17 mm², for example. Furthermore, in this embodiment, the flow cross section of the second inflow or outflow space can then be approximately 26 mm². The ratio of the sum of the flow cross sections of all passage openings to the flow cross section of the at least one second inflow or outflow space is 0.65:1.

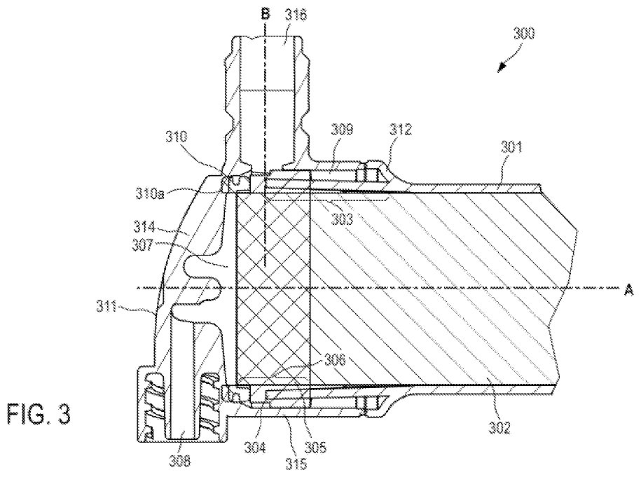

FIG. 3 shows a schematic representation of a cross section of a commercially available FX hollow fiber membrane filter from Fresenius Medical Care Deutschland GmbH, which runs through both the central axis A of the cylindrical housing and the central axis B of the second liquid access point. Analogously to the previous figures, FIG. 3 shows:
301 a cylindrical case
302 a housing interior space of the cylindrical housing for receiving a plurality of hollow fiber membranes (not shown in FIG. 3)
303 an end region of the cylindrical housing
304 a first end of the cylindrical housing
305 a potting compound,
306 a potting zone,
307 a first inflow or outflow space,
308 a first liquid access point to the first inflow or outflow space,
309 a second inflow or outflow space,
310 a circumferential seal, embodied as an O-ring,
310a an inner circumference in the end cap,
311 an end cap,
312a an annular outer circumferential projection,
314 a wall of the end cap,
315 a casing of the end region of the cylindrical housing on the end cap,
316 a second liquid access point.

As can be seen from FIG. 3, the hollow fiber membrane filters shown in FIGS. 1a, 1b, and 3 differ structurally in terms of the construction of the second inflow and outflow space. The passage openings that connect the second inflow or outflow spaces to the second flow region of the hollow fiber membrane filter (not shown) are not visible in FIG. 3.

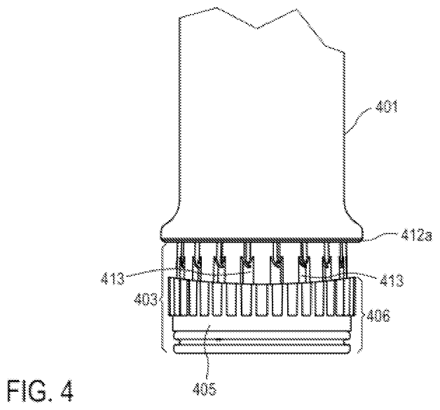

FIG. 4 shows a schematic representation of a side view of a cylindrical housing 401 of a commercially available FX hollow fiber membrane filter from Fresenius Medical Care Deutschland GmbH, which has a potting compound 405 in a potting zone 406. FIG. 4 shows an annular, outer circumferential projection 412a. The side view also shows the passage openings 413, which are arranged circumferentially on the end region 403 of the housing 401. The FX60 hollow fiber membrane filter illustrated according to FIGS. 3 and 4 has a flow cross section of the second inflow or outflow space of 26 mm². In the same embodiment of the FX hollow fiber membrane filter, the sum of the flow cross sections of all passage openings is 392 mm². The ratio of the sum of the flow cross sections of all passage openings to the flow cross section of the at least one second inflow or outflow space is 15:1.

### EXAMPLES

### Determination of Clearance

The clearance is determined in accordance with the DIN/EN/ISO 8637:2014 standard, with a blood flow of 300 ml/min and a dialysate flow of 500 ml/min being set in the examples. Aqueous solutions of 16.7 mmol/l urea (Merck) and 36.7 μmol/l vitamin B12 (BCD Chemie, Biesterfeld) on the blood side and distilled water on the dialysate side are used as test solutions. The concentration of vitamin B12 is determined photometrically at 361 nm. The Cobas Integra 400 plus device with the UREAL test (Roche Diagnostics, Germany) is used to determine the urea.

### Example 1: Hollow Fiber Membrane Filter According to the Invention

A hollow fiber membrane filter with the structural details according to FIGS. 1a and 1b and the parameters shown in Table 1 was produced. Corrugated polysulfone/polyvinylpyrrolidone hollow fiber membranes were used, which are particularly built into the FX60 filter from Fresenius Medical Care. The hollow fiber membrane filter was manufactured according to methods known in the prior art.

The hollow fiber membrane filter according to the invention was sterilized using a steam sterilization method that is known in the prior art and is described in application laid open DE 10 2016 224 627 A1. Clearance and sieve coefficients were examined on the sterile as well as on the non-sterile embodiment. The results are shown in Table 2.

### Comparative Example 1: FX60 Hollow Fiber Membrane Filter

An FX60 hollow fiber membrane filter from Fresenius Medical Care was used as a comparative embodiment. The structural details of the FX 60 hollow fiber membrane filter are shown schematically in FIGS. 3 and 4. The technical parameters of the FX60 filter are shown in Table 1.

The FX60 hollow fiber membrane filter was sterilized using the same steam sterilization process that was used for the hollow fiber membrane filter according to the invention. The clearance determined using the hollow fiber membrane filter was examined on the sterile as well as on the non-sterile embodiment. The results are shown in Table 2.

TABLE 1

| | | | | Comparative | |
|---|---|---|---|---|---|
| Parameter | Characteristic | Example 1 | | example 1 | |
| 1 | Number of hollow fiber | 8448 | | 10752 | |
| | membranes | | | | |
| 2 | Actual effective length of | 285 | mm | 228 | mm |
| | hollow fiber membranes | | | | |
| 3 | Membrane surface area | 1.4 | m² | 1.4 | m² |
| 4 | Inner diameter of hollow | 184 | μm | 184 | μm |
| | fiber membranes | | | | |
| 5 | Wall thickness of hollow fiber | 37 | μm | 37 | μm |
| | membranes | | | | |
| 6 | Amplitude of hollow fiber | 0.41 | mm | 0.41 | mm |
| | membranes | | | | |
| 7 | Wavelength | 7.5 | mm | 7.5 | mm |
| 8 | Inner diameter of cylindrical | 31 | mm | 34 | mm |
| | housing | | | | |
| 9 | Σ Flow cross sections of all | 24.1 | mm² | 315.3 | mm² |
| | passage openings | | | | |
| 10 | Flow cross section of the | 23.6 | mm² | 26.4 | mm² |
| | second inflow or outflow | | | | |
| | space | | | | |
| 11 | Quotient from parameters 9 | 1.02:1 | | 11.9:1 | |
| | and 10 | | | | |

| | | | |
|---|---|---|---|
| 12 | Aspect ratio | 9.19 | 6.71 |

Hollow fiber membranes originating from the same production were used for the hollow fiber membrane filter according to the invention according to Example 1 and for the FX 60 hollow fiber membrane filter according to Comparative Example 1. These hollow fiber membranes match in terms of diameter, wall thickness, pore properties, and material composition. The number of hollow fiber membranes in Example 1 and Comparative Example 1 was adjusted so that the respective hollow fiber membrane filters each had the same membrane surface area of 1.4 m².

TABLE 2

| | Ex. 1, | Comp. Ex. 1, | Ex. 1, non- | Comp. Ex. 1, |
|---|---|---|---|---|
| | sterile | sterile | sterile | non-sterile |
| Clearance, | 273 ml/min | 267 ml/min | 276 ml/min | 274 ml/min |
| urea | | | | |
| Clearance, | 175 ml/min | 169 ml/min | 176 ml/min | 169 ml/min |
| Vit. B12 | | | | |

The results from Table 2 show that the clearance of sterile and non-sterile hollow fiber membrane filters according to Example 1 for urea and vitamin B12 is higher than for the FX60 hollow fiber membrane filter of Comparative Example 1. In addition, the example according to the invention shows only a slight decrease in urea clearance after sterilization.

1. A hollow fiber membrane filter, comprising
a cylindrical housing that extends along a central axis in the longitudinal direction, with a housing interior space, a first end region with a first end, and a second end region with a second end,
a plurality of hollow fiber membranes that are arranged in the cylindrical housing and embedded in a sealing manner in the first end region and in the second end region of the cylindrical housing in a respective potting compound in a potting zone, the ends of the hollow fiber membranes being open so that the lumina the hollow fiber membranes form a first flow space and the housing interior space surrounding the hollow fiber membranes forms a second flow space,
first inflow or outflow spaces, each adjoining with their front end the first and second end of the cylindrical housing and the potting zone, which are in fluid communication with the first flow space of the hollow fiber membrane filter and each of which has first liquid access points for conducting liquid into/out of the first inflow or outflow spaces,
second inflow or outflow spaces surrounding the first and the second end region of the cylindrical housing which are in fluid communication with the second flow region and each of which has second liquid access points for conducting liquid into/out of the second inflow or outflow space,
a respective seal that separates the first inflow or outflow spaces from the second inflow or outflow spaces,
passage openings in the end regions of the cylindrical housing that form a fluid connection between the second inflow and/or outflow spaces and the second flow space, wherein
in at least one end region of the cylindrical housing, the ratio of the sum of the flow cross sections of all passage openings to the flow cross section of the at least one second inflow or outflow space is between 0.5:1 to 7:1.

2. The hollow fiber membrane filter as set forth in claim 1, wherein in the at least one end region of the cylindrical housing in which the ratio of the sum of the flow cross sections of all passage openings to the flow cross section of the at least one second inflow or outflow space is present, the at least one second inflow or outflow space, starting from the second liquid access point, forms a circumferential space that is rotationally symmetrical to the central axis of the cylindrical housing.

3. The hollow fiber membrane filter as set forth in claim 2, wherein both second inflow or outflow spaces form the rotationally symmetrical circumferential space.

4. The hollow fiber membrane filter as set forth in claim 1, wherein said at least one end region, and optionally said second end region, is divided into a proximal end region, a distal end region, and a transition region disposed between said proximal and distal end regions, wherein one end of the distal end regions of the first and/or second end region corresponds to the respective end of the cylindrical housing, and the distal end region has an inner diameter at least 2% larger than the inner diameter of the proximal end region.

5. The hollow fiber membrane filter as set forth in claim 4, wherein the passage openings are arranged at the distal end region.

6. The hollow fiber membrane filter as set forth in claim 1, wherein the passage openings are circular, oval-, or slot-shaped.

7. The hollow fiber membrane filter as set forth in claim 1, wherein the passage openings are arranged on isolated and/or opposite sections or circumferentially on the end region of the cylindrical housing.

8. The hollow fiber membrane filter as set forth in claim 1, wherein the sum of the flow cross sections of all passage openings is 10 to 350 mm².

9. The hollow fiber membrane filter as set forth in claim 1, wherein the flow cross section of the one second or two second inflow or outflow spaces is 20 to 50 mm².

10. The hollow fiber membrane filter as set forth in claim 1, wherein the first and the second inflow or outflow space in the first end region of the cylindrical housing and the first and the second inflow or outflow space in the second end region of the cylindrical housing are respectively enclosed by a first and a second end cap.

11. The hollow fiber membrane filter as set forth in claim 8, wherein the first and the second end cap adjoin an annular outer circumferential projection on the first and on the second end region of the cylindrical housing in a positive, liquid-tight manner.

12. The hollow fiber membrane filter as set forth in claim 8, wherein the first and the second end cap positively adjoin the first end and the second end, respectively, of the cylindrical housing in a liquid-tight manner, along an inner circumferential circular line.

13. The hollow fiber membrane filter as set forth in claim 1, wherein, in the end regions in the vicinity of the passage openings, the cylindrical housing has an inner diameter of 20 to 45 mm.

14. The hollow fiber membrane filter as set forth in claim 1, wherein the aspect ratio of the hollow fiber membrane filter is 8 to 12.

15. The hollow fiber membrane filter of claim 1, wherein the ratio of the sum of the flow cross sections of all passage openings to the flow cross section of the at least one second inflow or outflow space is between 0.75:1 to 5:1.

16. The hollow fiber membrane filter of claim 1, wherein the ratio of the sum of the flow cross sections of all passage openings to the flow cross section of the at least one second inflow or outflow space is between 1:1 to 3:1.

17. The hollow fiber membrane filter of claim 2, wherein the circumferential space is an annular gap.

18. The hollow fiber membrane filter of claim 8, wherein the sum of the flow cross sections of all passage openings is 15 to 200 mm².

19. The hollow fiber membrane filter of claim 8, wherein the sum of the flow cross sections of all passage openings is 15 to 150 mm².

20. The hollow fiber membrane filter of claim 8, wherein the sum of the flow cross sections of all passage openings is 20 to 110 mm².
